(19) United States
(12) Patent Application Publication
(10) Pub. No.: US 2025/0154095 A1
(43) Pub. Date: May 15, 2025

(54) SYNTHESIS METHOD OF AVENANTHRAMIDE C

(71) Applicant: Dong-A University Research Foundation for Industry-Academy Cooperation, Busan (KR)

(72) Inventors: Jong Hyun CHO, Gimhae-si (KR); Se Myeong CHOI, Busan (KR); Ye Eun NAM, Busan (KR); Yeon Jin AN, Ulsan (KR); Eun Rang CHOI, Busan (KR); Eun Woo SEO, Changwon-si (KR)

(73) Assignee: Dong-A University Research Foundation for Industry-Academy Cooperation, Busan (KR)

(21) Appl. No.: 18/730,530

(22) PCT Filed: May 6, 2022
(86) PCT No.: PCT/KR2022/006471

§ 371 Date: Jul. 19, 2024

(30) Foreign Application Priority Data
Jan. 20, 2022 (KR) 10-2022-0008430

(51) Int. Cl. C07C 231/02; C07C 227/16; C07C 231/24

(52) U.S. Cl. CPC C07C 231/02; C07C 227/16; C07C 231/24

(57) ABSTRACT

The present disclosure relates to a synthesis method of Avenanthramides, and more particularly, to a new large-scale synthesis method capable of obtaining Avenanthramide C with high yield through a pathway of introducing a protecting group to starting materials, 2-Amino-5-hydroxy benzoic acid and Caffeic acid, synthesizing the compound introduced with the protecting group through a nucleophilic substitution (S_{N}2) reaction in the presence of oxalyl chloride ((COCl)₂) and N,N-dimethylformamide (DMF), and removing the protecting group.

### TECHNICAL FIELD

The present disclosure relates to a synthesis method of avenanthramides, and more particularly, to a new large-scale synthesis method capable of obtaining avenanthramide C with high yield through a pathway of introducing a protecting group to starting materials, 2-amino-5-hydroxy benzoic acid and caffeic acid, synthesizing the compound introduced with the protecting group through a nucleophilic substitution (S_{N}2) reaction in the presence of oxalyl chloride ((COCl)₂) and N,N-dimethylformamide (DMF), and removing the protecting group.

### BACKGROUND ART

Oats contain various types of polyphenols, and among them, avenanthramides are specific antioxidant components extracted from the oats, and are effective against atopic dermatitis, known to be powerful antioxidants, and reported to have potential anti-inflammatory and blood pressure regulating effects. Recently, the oats have been found to have a therapeutic effect on Alzheimer's dementia, noise-induced hearing loss, and drug-induced hearing loss, and an excellent preventive effect on anticancer drug-induced hearing loss.

Previously, in order to use the avenanthramides as an active ingredient, research has been conducted on methods for increasing the content of avenanthramides in oats or extracting avenanthramides from oats. For example, in Korean Patent Registration No. 10-1745734, there is disclosed a method for preparing oats having a concentration of avenanthramides increased by pre-germination.

After avenanthramides (Avn) are first found, attempts have been made to isolate and purify the Avn from oats, but have not been completely successful.

As a natural product of avenanthramides extracted from oats, there are limitations in securing large quantities of materials for clinical trials and enormous costs are required for research and development of new drugs. Previously reported synthetic methods for avenanthramides were not suitable for large-scale synthesis due to low yield caused by various by-products, unmanageable reagents, and difficulties in separation and purification. Currently, no effective large-scale synthesis process for avenanthramides has been reported.

In order to study the wide range of drug effects of avenanthramides, including anti-inflammatory, anticancer, dementia prevention, and hearing loss-prevention effects, it is necessary to secure an effective synthetic method.

### PRIOR ARTS

### Patent Documents

Korean Patent Registration No. 10-1745734

### DISCLOSURE

### Technical Problem

An object of the present disclosure is to provide a synthesis method of avenanthramide C including, as illustrated in the following Reaction Formula A, Step 1 of reacting Compound 1 in the presence of methyl alcohol and an acid catalyst to obtain Compound 2 having a methyl protecting group introduced to a carbonyl group; Step 2 of reacting Compound 3 and acetic anhydride in the presence of a first organic solvent to obtain Compound 4 having an acetyl protecting group introduced to a hydroxyl group of benzene; Step 3 of reacting Compound 2 and Compound 4 in the presence of a second organic solvent to obtain Compound 5; Step 4 of removing the acetyl protecting group from Compound 5 to obtain Compound 6; and Step 5 of removing the methyl protecting group from Compound 6 to obtain Compound 7.

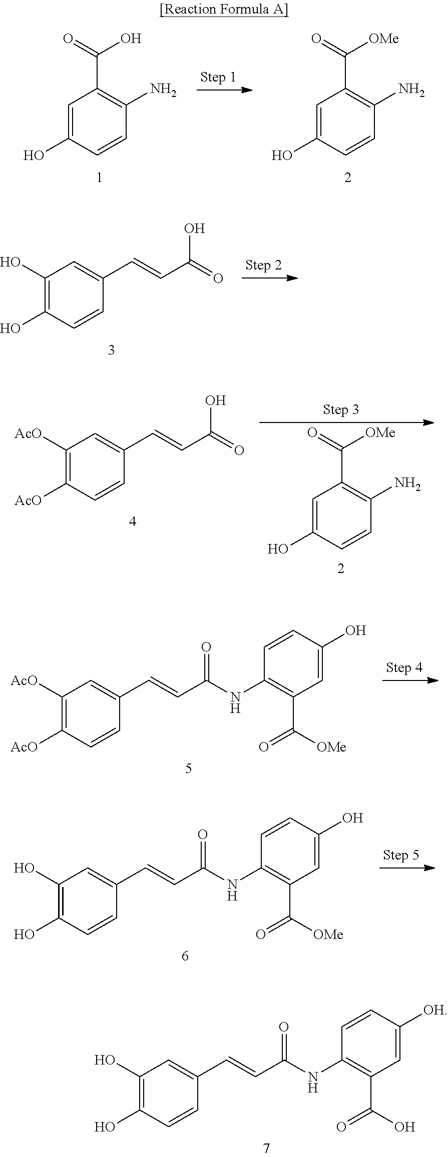

Another object of the present disclosure is to provide a synthesis method of methyl 2-amino-5-hydroxy benzoic acid represented by Chemical Formula 2 below, including reacting 2-amino-5-hydroxy benzoic acid represented by Chemical Formula 1 below and methyl alcohol in the presence of an acid catalyst:

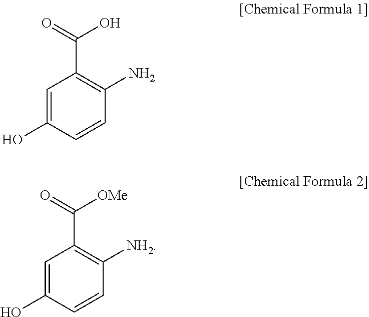 [SMILES: Nc1ccc(O)cc1C(=O)O ; COC(=O)c1cc(O)ccc1N]

### Technical Solution

In order to achieve the object,
an aspect of the present disclosure provides a synthesis method of avenanthramide C including, as illustrated in the following Reaction Formula A, Step 1 of reacting Compound 1 in the presence of methyl alcohol and an acid catalyst to obtain Compound 2 having a methyl protecting group introduced to a carbonyl group; Step 2 of reacting Compound 3 and acetic anhydride in the presence of a first organic solvent to obtain Compound 4 having an acetyl protecting group introduced to a hydroxyl group of benzene; Step 3 of reacting Compound 2 and Compound 4 in the presence of a second organic solvent to obtain Compound 5; Step 4 of removing the acetyl protecting group from Compound 5 to obtain Compound 6; and Step 5 of removing the methyl protecting group from Compound 6 to obtain Compound 7:

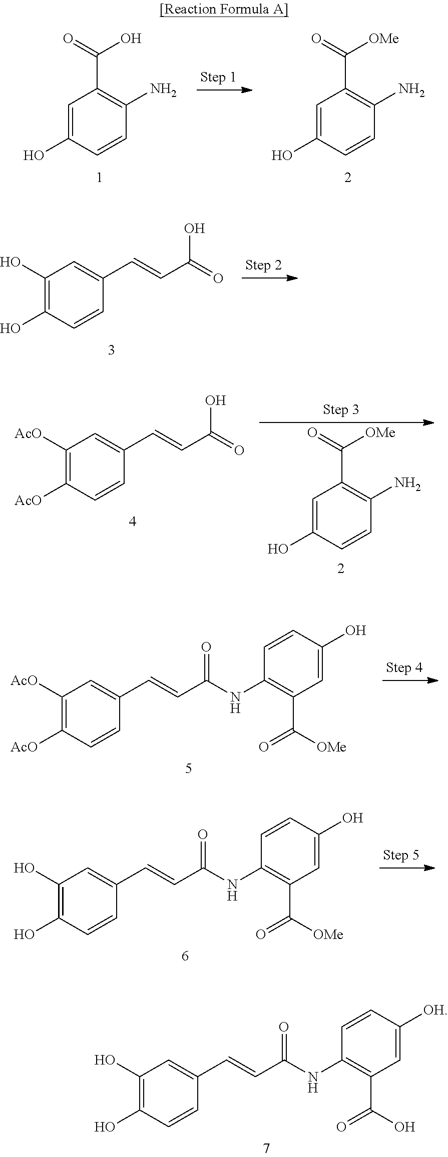

Another aspect of the present disclosure provides a synthesis method of methyl 2-amino-5-hydroxy benzoic acid represented by Chemical Formula 2 below, including reacting 2-amino-5-hydroxy benzoic acid represented by Chemical Formula 1 below and methyl alcohol in the presence of an acid catalyst:

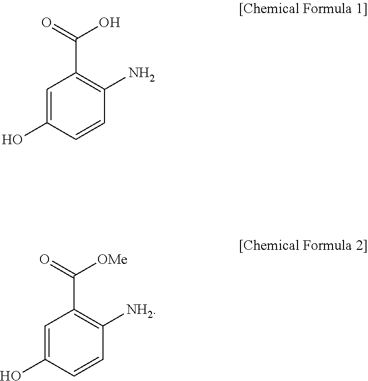 [SMILES: Nc1ccc(O)cc1C(=O)O ; COC(=O)c1cc(O)ccc1N]

### Advantageous Effects

According to the present disclosure, the synthesis method of avenanthramide C can be useful for a mass-production by using manageable reagents and synthesizing avenanthramide C with a high yield, compared to a conventional synthetic method. Accordingly, it is possible to secure and stably supply avenanthramide C for research on a wide range of drug effects, including anti-inflammation, anti-cancer, dementia prevention, and hearing loss prevention by overcoming the limitations of isolating trace amounts from a natural product, oats, to enable a stable supply of avenanthramides required for clinical research.

### Modes of the Invention

Hereinafter, the present disclosure will be described in detail.

An aspect of the present disclosure provides a synthesis method of avenanthramide C including, as illustrated in the following Reaction Formula A, Step 1 of reacting Compound 1 in the presence of methyl alcohol and an acid catalyst to obtain Compound 2 having a methyl protecting group introduced to a carbonyl group; Step 2 of reacting Compound 3 and acetic anhydride in the presence of a first organic solvent to obtain Compound 4 having an acetyl protecting group introduced to a hydroxyl group of benzene; Step 3 of reacting Compound 2 and Compound 4 in the presence of a second organic solvent to obtain Compound 5; Step 4 of removing the acetyl protecting group from Compound 5 to obtain Compound 6; and Step 5 of removing the methyl protecting group from Compound 6 to obtain Compound 7:

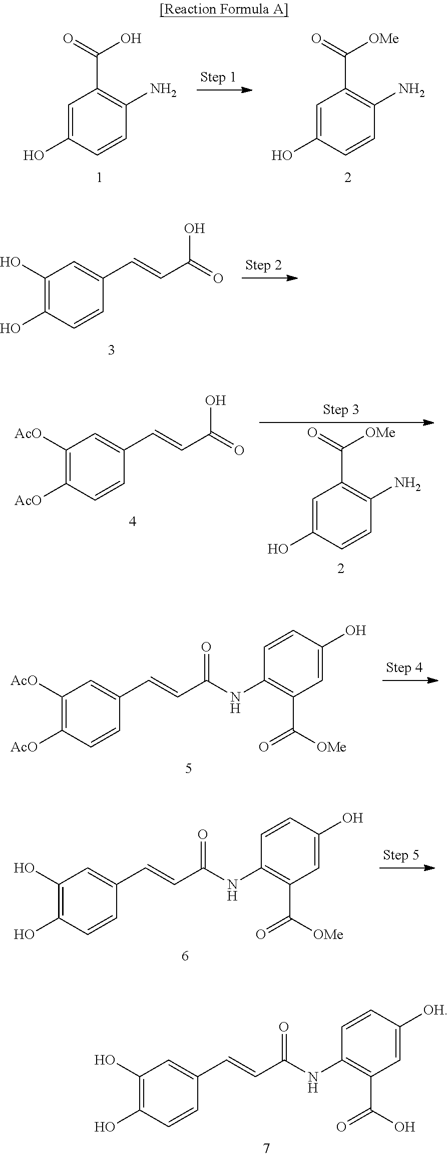

First, the synthesis method of the present disclosure includes Step 1 of reacting Compound 1 in the presence of methyl alcohol and an acid catalyst to obtain Compound 2 having a methyl protecting group introduced to a carbonyl group; and Step 2 of reacting Compound 3 and acetic anhydride in the presence of a first organic solvent to obtain Compound 4 having an acetyl protecting group introduced to a hydroxyl group of benzene.

In an embodiment of the present disclosure, Steps 1 and 2 may correspond to a process of introducing a protecting group to 2-amino-5-hydroxy benzoic acid and caffeic acid, which are starting materials for the synthesis of avenanthramides.

Through the process of introducing the protecting group to the 2-amino-5-hydroxy benzoic acid and the caffeic acid, thereafter, in the S_{N}2 reaction for the synthesis of avenanthramide C, the production of additional products is suppressed, thereby improving the yield of avenanthramide C.

In an embodiment of the present disclosure, the acid catalyst of Step 1 may be at least one of hydrochloric acid (HCl), nitric acid (HNO₃), acetic acid (CH₃COOH), perchloric acid (HClO₄), phosphoric acid (H₃PO₄), paratoluenesulfonic acid (p-TsOH), formic acid (HCO₂H), and sulfuric acid (H₂SO₄), for example, sulfuric acid (H₂SO₄).

In addition, in an embodiment of the present disclosure, Step 1 may be performed by refluxing for 42 hours to 54 hours, for example, 45 hours to 51 hours, for example, 47 hours to 49 hours.

The following Reaction Formula 1 illustrates a specific process of Step 1 of an embodiment of the present disclosure:

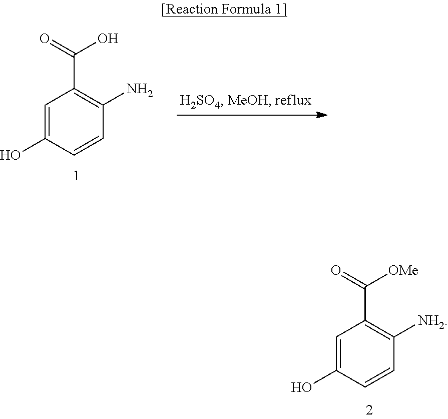 [SMILES: Nc1ccc(O)cc1C(=O)O ; COC(=O)c1cc(O)ccc1N]

Referring to Reaction Formula 1 above, when describing Step 1 of the synthesis method of the present disclosure in detail, methyl 2-amino-5-hydroxy benzoic acid with a methyl protecting group introduced to a carboxyl group of 2-amino-5-hydroxy benzoic acid may be synthesized by adding sulfuric acid (H₂SO₄) as an acid catalyst to 2-amino-5-hydroxy benzoic acid in the presence of a methyl alcohol solvent and refluxing for 47 to 49 hours.

In an embodiment of the present disclosure, in Step 2, the first organic solvent may be any one selected from the group consisting of dichloromethane (CH₂Cl₂), 1,2-dichloroethane (CH₂ClCH₂Cl), tetrahydrofuran (THF), dimethyl sulfoxide (DMSO), t-butyl methyl ether (TBME), acetonitrile (ACN), methyl alcohol, ethyl alcohol, isopropyl alcohol, t-butanol, diethyl ether, diphenyl ether, diisopropyl ether (DIPE), dimethyl formamide (DMF), dimethyl acetamide (DMA), chlorobenzene, benzene, toluene, carbon tetrachloride (CCl₄), acetone, trifluoroacetic acid, chloroform (CHCl₃), pyridine, and aqueous solutions thereof, or a mixed solution thereof, and for example, pyridine.

In an embodiment of the present disclosure, Step 2 may be performed by stirring at room temperature for 18 hours to 30 hours, for example, 21 hours to 37 hours, for example, 23 hours to 25 hours.

The following Reaction Formula 2 illustrates a specific process of Step 2 of an embodiment of the present disclosure:

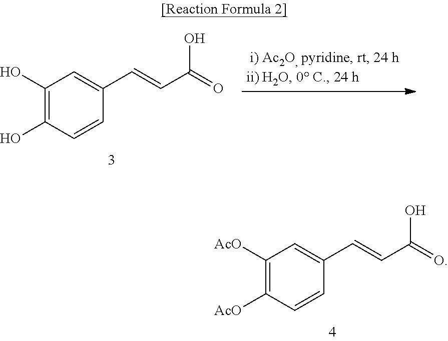 [SMILES: O=C(O)/C=C/c1ccc(O)c(O)c1 ; CC(=O)Oc1ccc(/C=C/C(=O)O)cc1OC(C)=O]

Referring to Reaction Formula 2 above, when describing Step 2 of the synthesis method of the present disclosure in detail, (E)-3-(3,4-diacetoxyphenyl) acrylic acid may be obtained by sequentially adding pyridine and acetic anhydride to caffeic acid, reacting the mixture while stirring for 23 to 25 hours, and then synthesizing a product in which an acetyl protecting group is introduced to a hydroxyl group of benzene, and precipitating the product as a solid at about 0° C.

Next, the synthetic method of the present invention includes Step 3 of reacting Compound 2 and Compound 4 in the presence of a second organic solvent to obtain Compound 5.

In an embodiment of the present disclosure, the second organic solvent may be any one selected from the group consisting of dichloromethane (CH₂Cl₂), 1,2-dichloroethane (CH₂ClCH₂Cl), tetrahydrofuran (THF), dimethyl sulfoxide (DMSO), t-butyl methyl ether (TBME), acetonitrile (ACN), methyl alcohol, ethyl alcohol, isopropyl alcohol, t-butanol, diethyl ether, diphenyl ether, diisopropyl ether (DIPE), dimethyl formamide (DMF), dimethyl acetamide (DMA), chlorobenzene, benzene, toluene, carbon tetrachloride (CCl₄), acetone, trifluoroacetic acid, chloroform (CHCl₃), pyridine, and aqueous solutions thereof, or a mixed solution thereof, and for example, dichloromethane (CH₂Cl₂).

In an embodiment of the present disclosure, Step 3 may include Step 3-1 of adding and stirring oxalyl chloride ((COCl)₂) and dimethylformamide (DMF) to Compound 4; and Step 3-2 of adding Compound 2 to the mixture obtained in Step 3-1 above, and may be performed through a nucleophilic substitution reaction, for example, an S_{N}2 reaction of Compounds 2 and 4.

In an embodiment of the present disclosure, the nucleophilic substitution reaction may be performed at 50° C. to 70° C., for example, 55° C. to 65° C., for example, 58° C. to 63° C., for 10 minutes to 30 minutes, for example, 15 minutes to 25 minutes, for example, 18 minutes to 22 minutes.

In addition, Step 3 may further include a process of purifying the reactant of the nucleophilic substitution reaction using a silica gel chromatography purification method.

The following Reaction Formula 3 illustrates a specific process of Step 3 of an embodiment of the present disclosure:

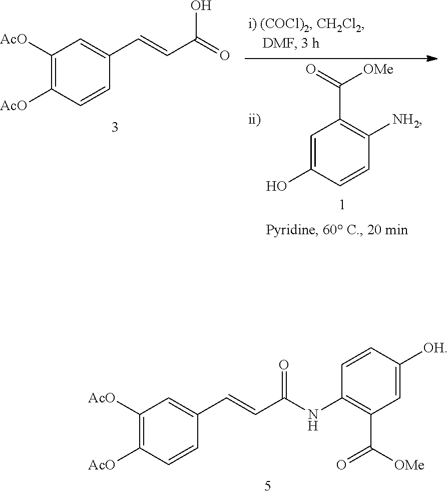 [SMILES: CC(=O)Oc1ccc(/C=C/C(=O)O)cc1OC(C)=O ; COC(=O)c1cc(O)ccc1N ; COC(=O)c1cc(O)ccc1NC(=O)/C=C/c1ccc(OC(C)=O)c(OC(C)=O)c1]

Referring to Reaction Formula 3 above, when describing Step 3 of the synthesis method of the present disclosure in detail, (E)-4-(3-((4-hydroxy-2-(methoxycarbonyl)phenyl)amino)-3-oxoprop-1-en-1-yl)-1,2-phenylene diacetate may be obtained by a method of adding and stirring (E)-3-(3,4-diacetoxyphenyl) acrylic acid with (COCl)₂ and N,N-dimethylformamide (DMF), slowly adding methyl 2-amino-5-hydroxy benzoic acid dissolved in pyridine, and then performing an S_{N}2 reaction at 58° C. to 63° C. for 18 to 22 minutes to obtain a reactant, and separating the reactant by a silica gel chromatography purification method.

Next, the synthesis method of the present disclosure includes Step 4 of removing the acetyl protecting group from Compound 5 to obtain Compound 6; and Step 5 of removing the methyl protecting group from Compound 6 to obtain Compound 7.

In an embodiment of the present disclosure, Steps 4 and 5 may correspond to a protecting group removal process for removing the methyl protecting group and the acetyl protecting group introduced in Steps 1 and 2, respectively.

In an embodiment of the present disclosure, Step 4 may be performed by adding any one of NaH, KH, LiOH, NaOH, KOH, Ca(OH)₂, Mg(OH)₂, NaOMe, NaOEt, Na₂CO₃, NaHCO₃, NH₃, Et₃N, DIEA, DMAP, pyridine, CsOH, Cs₂CO₃, KHCO₃ and K₂CO₃ as a base, for example, NaOMe, in the presence of a third organic solvent, and stirring at room temperature for 1 hour to 3 hours, for example, 1.5 hours to 2.5 hours, for example, 1.8 hours to 2.2 hours.

In an embodiment of the present disclosure, the third organic solvent may be any one selected from the group consisting of dichloromethane (CH₂Cl₂), 1,2-dichloroethane (CH₂CCH₂Cl), tetrahydrofuran (THF), dimethyl sulfoxide (DMSO), t-butyl methyl ether (TBME), acetonitrile (ACN), methyl alcohol, ethyl alcohol, isopropyl alcohol, t-butanol, diethyl ether, diphenyl ether, diisopropyl ether (DIPE), dimethyl formamide (DMF), dimethyl acetamide (DMA), chlorobenzene, benzene, toluene, carbon tetrachloride (CCl₄), acetone, trifluoroacetic acid, chloroform (CHCl₃), pyridine, and aqueous solutions thereof, or a mixed solution thereof, and for example, a mixed solution of methyl alcohol and acetone.

The following Reaction Formula 4 illustrates a specific process of Step 4 of an embodiment of the present disclosure:

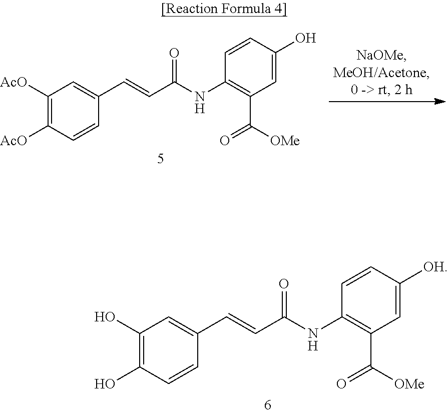 [SMILES: COC(=O)c1cc(O)ccc1NC(=O)/C=C/c1ccc(OC(C)=O)c(OC(C)=O)c1 ; COC(=O)c1cc(O)ccc1NC(=O)/C=C/c1ccc(O)c(O)c1]

Referring to Reaction Formula 4 above, when describing Step 4 of the synthesis method of the present disclosure in detail, methyl (E)-2-(3-(3,4-dihydroxyphenyl) acrylamido)-5-hydroxybenzoate from which the acetyl protecting group of benzene is removed may be obtained by adding sodium methoxide (NaOMe) to (E)-4-(3-((4-hydroxy-2-(methoxycarbonyl)phenyl)amino)-3-oxoprop-1-en-1-yl)-1,2-phenylene diacetate in the presence of a methyl alcohol/acetone solvent at about 0° C., stirring at room temperature for 1.8 hours to 2.2 hours to produce a reactant, and separating the reactant by a silica gel chromatography purification method.

In an embodiment of the present disclosure, Step 5 may be performed by adding any one of NaH, KH, NaOH, KOH, Ca(OH)₂, Mg(OH)₂, NaOMe, NaOEt, Na₂CO₃, NaHCO₃, NH₃, Et₃N, DIEA, DMAP, pyridine, CsOH, Cs₂CO₃, KHCO₃ or K₂CO₃, and LiOH as a base, for example, LiOH, in the presence of a fourth organic solvent, and stirring at room temperature for 2 hour to 4 hours, for example, 2.5 hours to 3.5 hours, for example, 2.8 hours to 3.2 hours.

In an embodiment of the present disclosure, the fourth organic solvent may be any one selected from the group consisting of dichloromethane (CH₂Cl₂), 1,2-dichloroethane (CH₂ClCH₂Cl), tetrahydrofuran (THF), dimethyl sulfoxide (DMSO), t-butyl methyl ether (TBME), acetonitrile (ACN), methyl alcohol, ethyl alcohol, isopropyl alcohol, t-butanol, diethyl ether, diphenyl ether, diisopropyl ether (DIPE), dimethyl formamide (DMF), dimethyl acetamide (DMA), chlorobenzene, benzene, toluene, carbon tetrachloride (CCl₄), acetone, trifluoroacetic acid, chloroform (CHCl₃), pyridine, and aqueous solutions thereof, or a mixed solution thereof, and for example, a mixed solution of THF and water.

The following Reaction Formula 5 illustrates a specific process of Step 5 of an embodiment of the present disclosure:

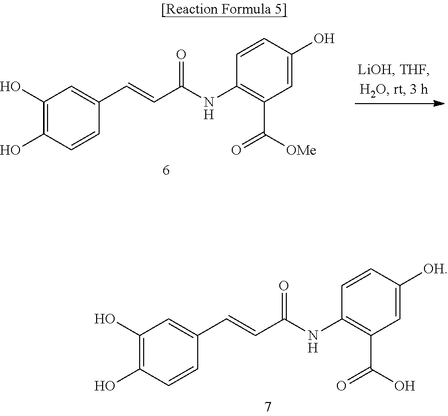 [SMILES: COC(=O)c1cc(O)ccc1NC(=O)/C=C/c1ccc(O)c(O)c1 ; O=C(/C=C/c1ccc(O)c(O)c1)Nc1ccc(O)cc1C(=O)O]

Referring to Reaction Formula 5 above, when describing Step 5 of the synthesis method of the present disclosure in detail, (E)-2-(3-(3,4-dihydroxyphenyl) acrylamido)-5-hydroxy benzoic acid (3-1) from which the methyl protecting group is removed, that is, avenanthramide C may be obtained by adding lithium hydroxide (LiOHH₂O) to methyl (E)-2-(3-(3,4-dihydroxyphenyl) acrylamido)-5-hydroxybenzoate in the presence of a THF/water solvent at about 0° C. and stirring and reacting at room temperature for 1.8 to 2.2 hours.

An aspect of the present disclosure provides a synthesis method of methyl 2-amino-5-hydroxy benzoic acid represented by Chemical Formula 2 below, including reacting 2-amino-5-hydroxy benzoic acid represented by Chemical Formula 1 below and methyl alcohol in the presence of an acid catalyst:

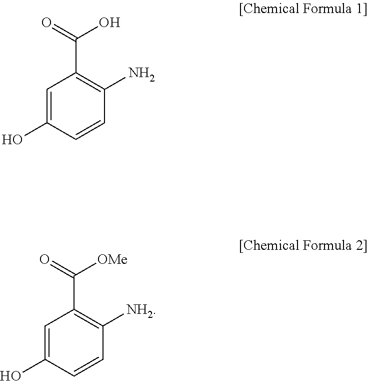 [SMILES: Nc1ccc(O)cc1C(=O)O ; COC(=O)c1cc(O)ccc1N]

In an embodiment of the present disclosure, the synthesis method of the methyl 2-amino-5-hydroxy benzoic acid may be the same as the process of Step 1 of the synthesis method of avenanthramide C of the aspect.

In an embodiment of the present disclosure, the acid catalyst may be at least one of hydrochloric acid (HCl), nitric acid (HNO₃), acetic acid (CH₃COOH), perchloric acid (HClO₄), phosphoric acid (H₃PO₄), paratoluenesulfonic acid (p-TsOH), formic acid (HCO₂H), and sulfuric acid (H₂SO₄), for example, sulfuric acid (H₂SO₄).

In addition, in an embodiment of the present disclosure, the synthesis method may be performed by refluxing for 42 hours to 54 hours, for example, 45 hours to 51 hours, for example, 47 hours to 49 hours.

The following Reaction Formula 1 illustrates a specific process of the synthesis method of an embodiment of the present disclosure:

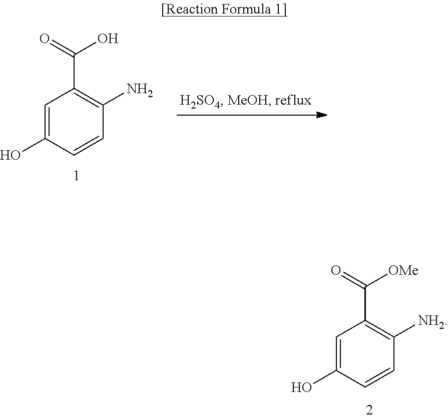 [SMILES: Nc1ccc(O)cc1C(=O)O ; COC(=O)c1cc(O)ccc1N]

Referring to Reaction Formula 1 above, when describing the synthesis method of the present disclosure in detail, methyl 2-amino-5-hydroxy benzoic acid with a methyl protecting group introduced to a carboxyl group of 2-amino-5-hydroxy benzoic acid represented by Compound 2 in Reaction Formula 1 above may be synthesized by adding sulfuric acid (H₂SO₄) as an acid catalyst to 2-amino-5-hydroxy benzoic acid represented by Compound 1 in Reaction Formula 1 in the presence of a methyl alcohol solvent and refluxing for 47 to 49 hours.

When using the synthesis method of the present disclosure, the methyl 2-amino-5-hydroxy benzoic acid may be synthesized from the 2-amino-5-hydroxy benzoic acid with a yield of about 90% or more.

In addition, as described above, the methyl 2-Amino-5-hydroxy benzoic acid synthesized by the synthesis method of the present disclosure may be used for synthesis of avenanthramide C of the aspect, as a form in which the methyl protecting group is introduced into a carbonyl group of 2-amino-5-hydroxy benzoic acid. The synthesis method of avenanthramides of the present disclosure can be used for a mass-production process by using manageable reagents and synthesizing avenanthramide with a high yield, compared to a conventional synthetic method that is unsuitable for a mass production process by using unmanageable reagents and with a low yield.

Accordingly, it is possible to secure and stably supply avenanthramides for research on a wide range of drug effects, including anti-inflammation, anti-cancer, dementia prevention, and hearing loss prevention by overcoming the limitations of isolating trace amounts from a natural product, oats, to enable a stable supply of avenanthramides required for clinical research.

Hereinafter, the present disclosure will be described in more detail through Examples. These Examples are to explain the present disclosure in more detail, and it will be apparent to those skilled in the art that the scope of the present disclosure is not limited to these Examples.

### EXAMPLES

### (1) Synthesis of methyl 2-Amino-5-hydroxy benzoic acid

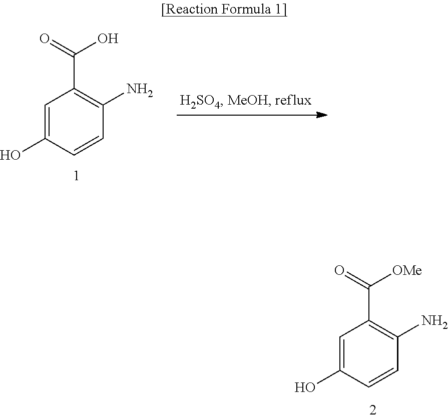 [SMILES: Nc1ccc(O)cc1C(=O)O ; COC(=O)c1cc(O)ccc1N]

In a 500 mL dried round flask, 2-amino-5-hydroxy benzoic acid (6 g, 39.2 mmol) was added in the presence of a methyl alcohol solvent, added with a catalytic amount of sulfuric acid (6 mL), and then refluxed and reacted for 48 hours. After the reaction was completed, the mixture was added with ice (10 g) and neutralized to pH 6 to 7 using a 1.0 N NaOH aqueous solution. After neutralization, the mixed aqueous solution was stirred at 0° C. for 1 hour, filtered, washed with cold water (10 mL×3), dried under reduced pressure for 1 hour, and then dried at room temperature for 12 hours in a vacuum state to synthesize methyl 2-amino-5-hydroxy benzoic acid as a brown solid in 90% yield.

1H NMR (400 MHZ, DMSO-d₆) δ8.67 (s, 1H), 7.10 (d, J=2.8 Hz, 1H), 6.82 (dd, J=2.8 Hz, 1H), 6.65 (d, J=8.8 Hz, 1H), 6.01 (br, 2H), 3.76 (s, 3H).

### (2) Synthesis of (E)-3-(3,4-diacetoxyphenyl) acrylic acid

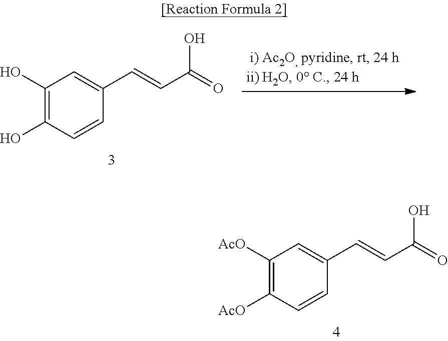 [SMILES: O=C(O)/C=C/c1ccc(O)c(O)c1 ; CC(=O)Oc1ccc(/C=C/C(=O)O)cc1OC(C)=O]

In a 250 mL dried round flask, caffeic acid (10 g, 55.5 mmol) as a starting material was sequentially added with pyridine (4 mL) and acetic anhydride (40 mL), stirred at room temperature for 24 hours, added with 50 mL of ice water, stirred for 30 minutes, and then refrigerated (4° C.) for 24 hours to precipitate a product as a white solid. The precipitated white solid was filtered, washed with cold water (20 mL×5), dried at room temperature under reduced pressure for 1 hour, and then dried in a vacuum state at 50° C. for 12 hours to synthesize (E)-3-(3,4-diacetoxyphenyl) acrylic acid in 95% yield.

1H NMR (400 MHZ, DMSO-d₆) δ 12.47 (br, 1H), 7.66 (d, J=1.6 Hz, 1H), 7.65 (d, J=1.6 Hz, 1H), 7.62 (d, J=1.6 Hz, 1H), 7.59 (d, J=16 Hz, 1H), 7.32 (d, J=8.4 Hz, 1H), 6.56 (d, J=16 Hz, 1H), 2.29 (s, 3H), 2.28 (s, 3H).

### (3) Synthesis of (E)-4-(3-((4-hydroxy-2-(methoxycarbonyl)phenyl)amino)-3-oxoprop-1-en-1-yl)-1,2-phenylene diacetate

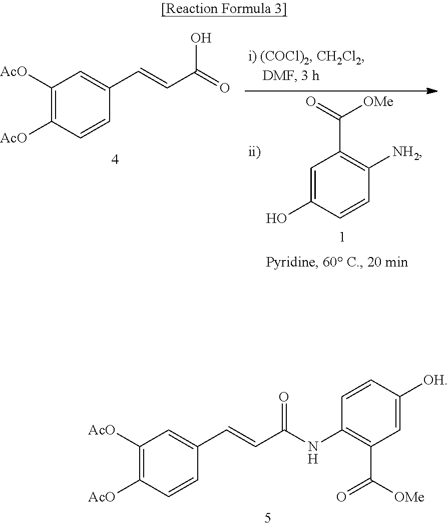 [SMILES: CC(=O)Oc1ccc(/C=C/C(=O)O)cc1OC(C)=O ; COC(=O)c1cc(O)ccc1N ; COC(=O)c1cc(O)ccc1NC(=O)/C=C/c1ccc(OC(C)=O)c(OC(C)=O)c1]

In a 250 mL dried round flask, (E)-3-(3,4-diacetoxyphenyl) acrylic acid) was added with (COCl)₂ (4.8 g, 37.8 mmol) and dimethylformamide (DMF) in a catalytic amount (0.01 mL), stirred at −10° C. for 3 hours, and slowly added with methyl 2-amino-5-hydroxy benzoic acid (2.87 g, 17.01 mmol) dissolved in pyridine (10 mL) at 0° C. for 20 minutes. After the addition was completed, the mixture was reacted at 60° C. for 20 minutes and then concentrated under reduced pressure. The residual mixture was adsorbed onto silica and then separated by a silica gel chromatography purification method (hexane:acetone=5:1->1:1) to synthesize (E)-4-(3-((4-hydroxy-2-(methoxycarbonyl)phenyl)amino)-3-oxoprop-1-en-1-yl)-1,2-phenylene diacetate in 65% yield (5.07 g, 12.3 mmol).

1H NMR (400 MHZ, DMSO-d₆) δ 10.47 (s, 1H), 9.72 (br, 1H), 8.14 (d, J=8.8 Hz, 1H), 7.69 (d, J=1.6 Hz, 1H), 7.65 (dd, J=2 Hz, 1H), 7.58 (d, J=15.6 Hz, 1H), 7.31-7.34 (m, 2H), 7.06 (dd, J=2.8 Hz), 6.92 (d, J=15.6 Hz, 1H), 3.85 (s, 3H), 2.30 (s, 3H), 2.22 (s, 3H).

### (4) Synthesis of methyl (E)-2-(3-(3,4-dihydroxyphenyl) acrylamido)-5-hydroxybenzoate

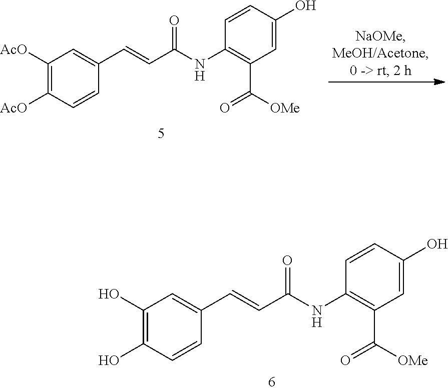 [SMILES: COC(=O)c1cc(O)ccc1NC(=O)/C=C/c1ccc(OC(C)=O)c(OC(C)=O)c1 ; COC(=O)c1cc(O)ccc1NC(=O)/C=C/c1ccc(O)c(O)c1]

In a 500 mL dried round flask, (E)-4-(3-((4-hydroxy-2-(methoxycarbonyl)phenyl)amino)-3-oxoprop-1-en-1-yl)-1,2-phenylene diacetate (5 g, 12 mmol) was added in the presence of a methyl alcohol/acetone (1:1 v/v, 200 mL) solvent, added with 0.5 mL of NaOMe at 0° C., stirred at room temperature for 2 hours, and then filtered and concentrated under reduced pressure, and the mixture was adsorbed onto silica and then separated by a silica gel chromatography purification method (CH₂Cl₂:acetone=10:1->1:1 v/v) to synthesize methyl (E)-2-(3-(3,4-dihydroxyphenyl) acrylamido)-5-hydroxybenzoate (3.7 g, 11.3 mmol) in 94% yield.

1H NMR (400 MHZ, DMSO-d₆) δ 10.32 (s, 1H), 9.66 (s, 1H), 9.47 (br, 1H), 9.12 (br, 1H), 8.07 (d, J=9.2 Hz, 1H), 7.4 (d, J=15.6 Hz, 1H), 7.29 (d, J=3.2 Hz, 1H), 7.06 (d, J=2 Hz, 1H), 7.04 (dd, J=2.8 Hz, 1h), 6.98 (dd, J=2 Hz, 1H), 6.77 (d, J=4.4 Hz, 1H), 6.54 (d, J=15.6 Hz, 1H), 3.83 (s, 3H).

### (5) Synthesis of (E)-2-(3-(3,4-dihydroxyphenyl) acrylamido)-5-hydroxy benzoic acid (avenanthramide C, Avn C)

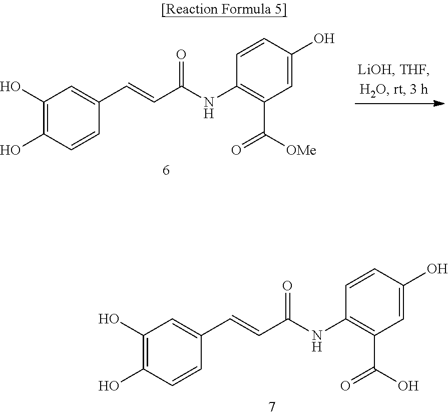 [SMILES: COC(=O)c1cc(O)ccc1NC(=O)/C=C/c1ccc(O)c(O)c1 ; O=C(/C=C/c1ccc(O)c(O)c1)Nc1ccc(O)cc1C(=O)O]

methyl (E)-2-(3-(3,4-dihydroxyphenyl) acrylamido)-5-hydroxybenzoate (5 g, 15.2 mmol) was added to a THF/water (1:1 v/v, 100 mL) solvent, added with LiOHH₂O (6.3 g, 152 mmol) at 0° C., and stirred at room temperature for 3 hours. The pH of the basic aqueous solution was lowered by adding a concentrated HCl aqueous solution (12 N), and the organic solvent was removed under reduced pressure. A brown solid formed in the aqueous solution was filtered, washed with cold water (20 mL×2) to remove water-soluble impurities, and dried under reduced pressure for 2 hours.

The dried solid was completely dissolved in an acetone (100 mL) solvent and transferred to a round flask, and the organic solvent was removed under reduced pressure, and dried in vacuum at room temperature for 24 hours to obtain (E)-2-(3-(3,4-dihydroxyphenyl) acrylamido)-5-hydroxy benzoic acid (avenanthramide C, Avn C) (4.30 g, 13.7 mmol) as a brown solid in 90% yield.

1H NMR (400 MHZ, DMSO-d₆) δ 10.84 (br, 1H), 9.57 (s, 1H), 9.48 (s, 1H), 9.11 (s, 1H), 8.33 (d, J=8.8 Hz, 1H), 7.40-7.36 (m, 2H), 7.06 (d, J=2.0 Hz, 1H), 7.02-6.95 (m, 2H), 6.77 (d, J=8.0, 1H), 6.49 (d, J=15.2 Hz, 1H).

Hereinabove, the present disclosure has been described with reference to preferred embodiments thereof. It will be understood to those skilled in the art that the present disclosure may be implemented as modified forms without departing from an essential characteristic of the present disclosure. Therefore, the disclosed embodiments should be considered in an illustrative viewpoint rather than a restrictive viewpoint. The scope of the present disclosure is defined by the appended claims rather than by the foregoing description, and all differences within the scope of equivalents thereof should be construed as being included in the present disclosure.

### INDUSTRIAL APPLICABILITY

According to the present disclosure, the synthesis method of avenanthramide C can be useful for a mass-production of avenanthramide C by using manageable reagents and synthesizing avenanthramide C with a high yield, compared to a conventional synthetic method.

1. A synthesis method of avenanthramide C comprising: as illustrated in the following Reaction Formula A,
Step 1 of reacting Compound 1 in the presence of methyl alcohol and an acid catalyst to obtain Compound 2 having a methyl protecting group introduced to a carbonyl group;
Step 2 of reacting Compound 3 and acetic anhydride in the presence of a first organic solvent to obtain Compound 4 having an acetyl protecting group introduced to a hydroxyl group of benzene;
Step 3 of reacting Compound 2 and Compound 4 in the presence of a second organic solvent to obtain Compound 5;
Step 4 of removing the acetyl protecting group from the Compound 5 to obtain Compound 6; and
Step 5 of removing the methyl protecting group from the Compound 6 to obtain Compound 7:

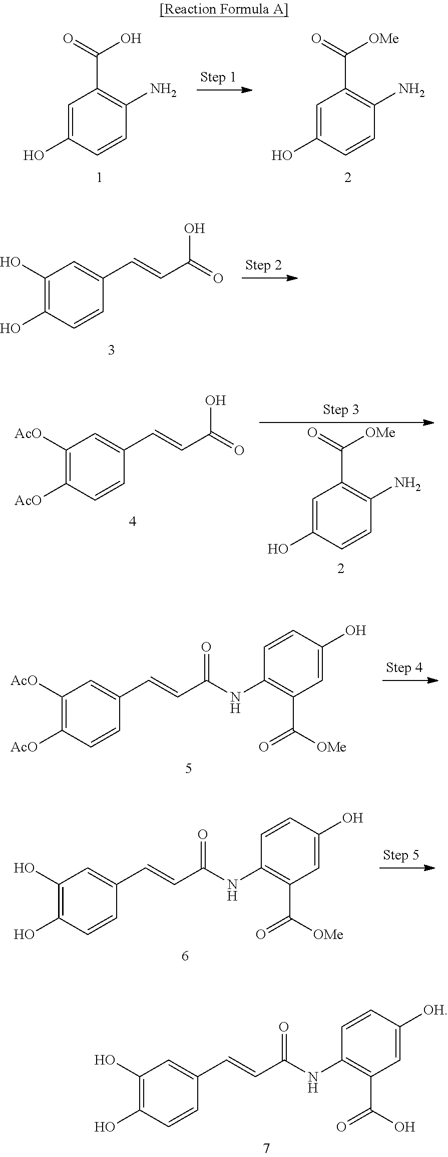

2. The synthesis method of claim 1, wherein the acid catalyst of the Step 1 is at least one of hydrochloric acid (HCl), nitric acid (HNO₃), acetic acid (CH₃COOH), sulfuric acid (H₂SO₄), perchloric acid (HClO₄), phosphoric acid (H₃PO₄), paratoluenesulfonic acid (p-TsOH), and formic acid (HCO₂H).

3. The synthesis method of claim 1, wherein the Step 1 is performed by refluxing for 42 hours to 54 hours.

4. The synthesis method of claim 1, wherein in the Step 2, the first organic solvent is any one selected from the group consisting of dichloromethane (CH₂Cl₂), 1,2-dichloroethane (CH₂ClCH₂Cl), tetrahydrofuran (THF), dimethyl sulfoxide (DMSO), t-butyl methyl ether (TBME), acetonitrile (ACN), methyl alcohol, ethyl alcohol, isopropyl alcohol, t-butanol, diethyl ether, diphenyl ether, diisopropyl ether (DIPE), dimethyl formamide (DMF), dimethyl acetamide (DMA), chlorobenzene, benzene, toluene, carbon tetrachloride (CCl₄), acetone, trifluoroacetic acid, chloroform (CHCl₃), pyridine, and aqueous solutions thereof, or a mixed solution thereof.

5. The synthesis method of claim 1, wherein the Step 2 is performed by stirring for 18 hours to 30 hours.

6. The synthesis method of claim 1, wherein in the Step 3, the second organic solvent is any one selected from the group consisting of dichloromethane (CH₂Cl₂), 1,2-dichloroethane (CH₂ClCH₂Cl), tetrahydrofuran (THF), dimethyl sulfoxide (DMSO), t-butyl methyl ether (TBME), acetonitrile (ACN), methyl alcohol, ethyl alcohol, isopropyl alcohol, t-butanol, diethyl ether, diphenyl ether, diisopropyl ether (DIPE), dimethyl formamide (DMF), dimethyl acetamide (DMA), chlorobenzene, benzene, toluene, carbon tetrachloride (CCl₄), acetone, trifluoroacetic acid, chloroform (CHCl₃), pyridine, and aqueous solutions thereof, or a mixed solution thereof.

7. The synthesis method of claim 1, wherein the Step 3 includes Step 3-1 of adding and stirring oxalyl chloride ((COCl)₂) and dimethylformamide (DMF) to the Compound 4; and
Step 3-2 of adding the Compound 2 to the mixture obtained in the Step 3-1 above.

8. The synthesis method of claim 1, wherein the Step 3 is performed by a nucleophilic substitution reaction of the Compound 2 and the Compound 4.

9. The synthesis method of claim 8, wherein the nucleophilic substitution reaction of the Step 3 is performed at 50° C. to 70° C. for 10 minutes to 30 minutes.

10. The synthesis method of claim 1, wherein the Step 3 further includes a process of purifying a reactant of the nucleophilic substitution reaction using a silica gel chromatography purification method.

11. The synthesis method of claim 1, wherein the Step 4 is performed by adding any one of NaH, KH, LiOH, NaOH, KOH, Ca(OH)₂, Mg(OH)₂, NaOMe, NaOEt, Na₂CO₃, NaHCO₃, NH₃, Et₃N, DIEA, DMAP, pyridine, CsOH, Cs₂CO₃, KHCO₃ and K₂CO₃ as a base, in the presence of a third organic solvent, and stirring at room temperature for 1 hour to 3 hours.

12. The synthesis method of claim 11, wherein the third organic solvent is any one selected from the group consisting of dichloromethane (CH₂Cl₂), 1,2-dichloroethane (CH₂ClCH₂Cl), tetrahydrofuran (THF), dimethyl sulfoxide (DMSO), acetonitrile (ACN), methyl alcohol, ethyl alcohol, isopropyl alcohol, t-butanol, diethyl ether, diphenyl ether, diisopropyl ether (DIPE), t-butyl methyl ether (TBME), dimethyl formamide (DMF), dimethyl acetamide (DMA), chlorobenzene, benzene, toluene, carbon tetrachloride (CCl₄), acetone, trifluoroacetic acid, chloroform (CHCl₃), pyridine, and aqueous solutions thereof, or a mixed solution thereof.

13. The synthesis method of claim 11, wherein the base is NaOMe.

14. The synthesis method of claim 1, wherein the Step 5 is performed by adding any one of NaH, KH, NaOH, KOH, Ca(OH)₂, Mg(OH)₂, NaOMe, NaOEt, Na₂CO₃, NaHCO₃, NH₃, Et₃N, DIEA, DMAP, pyridine, CsOH, Cs₂CO₃, KHCO₃ or K₂CO₃, and LiOH as a base, in the presence of a fourth organic solvent, and stirring at room temperature for 2 hour to 4 hours.

15. The synthesis method of claim 14, wherein the fourth organic solvent is any one selected from the group consisting of dichloromethane (CH₂Cl₂), 1,2-dichloroethane (CH₂ClCH₂Cl), tetrahydrofuran (THF), dimethyl sulfoxide (DMSO), t-butyl methyl ether (TBME), acetonitrile (ACN), methyl alcohol, ethyl alcohol, isopropyl alcohol, t-butanol, diethyl ether, diphenyl ether, diisopropyl ether (DIPE), dimethyl formamide (DMF), dimethyl acetamide (DMA), chlorobenzene, benzene, toluene, carbon tetrachloride (CCl₄), acetone, trifluoroacetic acid, chloroform (CHCl₃), pyridine, and aqueous solutions thereof, or a mixed solution thereof.

16. The synthesis method of claim 14, wherein the base is LiOH.

17. A synthesis method of methyl 2-amino-5-hydroxy benzoic acid represented by Chemical Formula 2 below, comprising reacting 2-amino-5-hydroxy benzoic acid represented by Chemical Formula 1 below and methyl alcohol in the presence of an acid catalyst:

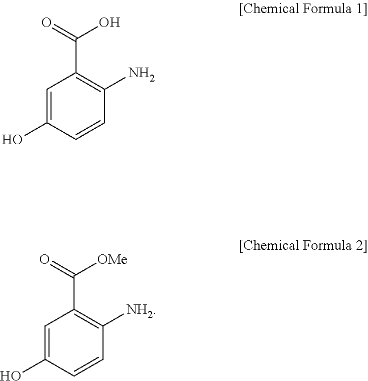 [SMILES: Nc1ccc(O)cc1C(=O)O ; COC(=O)c1cc(O)ccc1N]

18. The synthesis method of claim 17, wherein the acid catalyst is at least one of hydrochloric acid (HCl), nitric acid (HNO₃), acetic acid (CH₃COOH), sulfuric acid (H₂SO₄), perchloric acid (HClO₄), phosphoric acid (H₃PO₄), paratoluenesulfonic acid (p-TsOH), and formic acid (HCO₂H).

19. The synthesis method of claim 17, wherein the synthesis method is performed by refluxing for 42 hours to 54 hours.
